(19) United States
(12) Patent Application Publication
(10) Pub. No.: US 2023/0184575 A1
(43) Pub. Date: Jun. 15, 2023

(54) SYSTEMS AND METHODS FOR DETECTING FILL-LEVELS IN CROP TRANSPORT RECEPTACLES USING CAPACITANCE-BASED SENSOR ASSEMBLIES

(71) Applicant: CNH Industrial America LLC, New Holland, PA (US)

(72) Inventor: Scott Glovier, Lombard, IL (US)

(21) Appl. No.: 17/550,491

(22) Filed: Dec. 14, 2021

(51) Int. Cl. G01F 23/26; G01N 33/02; G01N 27/22

(52) U.S. Cl. CPC G01F 23/26; G01N 33/025; G01N 27/223

(57) ABSTRACT

In one aspect, a system for monitoring crop fill-levels of transport receptacles includes a crop transport receptacle defining a storage volume configured to receive harvested crops, and a sensor assembly provided in association with the crop transport receptacle. The sensor assembly is configured to generate capacitance-related data associated with a fill-level of the harvested crops within the storage volume. In addition, the system includes a computing system communicatively coupled to the sensor assembly. The computing system is configured to receive crop data associated with a dielectric constant of the harvested crops within the storage volume. The computing system is further configured to determine the fill-level of the harvested crops within the storage volume based at least in part on the capacitance-related data and the crop data.

### FIELD OF THE INVENTION

The present disclosure relates generally to crop transport receptacles and related transport vehicles for receiving crops during an unloading operation performed in association with a harvester and, more particularly, to systems and methods for detecting the fill-level of a crop transport receptacle using capacitance-based sensor assemblies.

### BACKGROUND OF THE INVENTION

Harvesters or harvesting machines pick up crop material, treat the crop material, e.g., remove any undesirable portions or residue, and discharge the crop material. Harvesters can discharge the crop material, either continuously as with a forage harvester or after intermediate storage as with a combine harvester, to a transport or transfer vehicle. The transport vehicle may be a tractor or truck pulling a cart, wagon, or trailer, or a truck or other vehicle capable of transporting harvested crop material. The harvested crop material is loaded into the transport vehicle via a crop discharging or unloading device, such as a spout or discharge auger, associated with the harvester.

During the performance of an unloading operation from a harvester to a transport vehicle, it is generally desirable to monitor the fill-level of the associated receptacle of the transport vehicle. In this regard, various vision-based systems have been proposed that utilize cameras to capture images of the harvested crops within the receptacle and then subsequently employ computer-vision techniques to process the images in an attempt to calculate or estimate the fill-level of the receptacle. However, such vision-based systems are often very complex and expensive and typically require significant computing resources to process and analyze the images in an efficient manner, particularly for “on-the-go” unloading operations.

Accordingly, systems and methods for monitoring the fill-level of a crop transport receptacle that address one or more of the issues present in the prior art would be welcomed in the technology, including, for example, systems and methods that provide a simpler (e.g., including less resource intensive) and/or more cost effective means for monitoring the fill-level within crop transport vehicles.

### SUMMARY OF THE INVENTION

Aspects and advantages of the technology will be set forth in part in the following description, or may be obvious from the description, or may be learned through practice of the technology.

In one aspect, the present subject matter is directed to a system for monitoring crop fill-levels of transport receptacles. The system includes a crop transport receptacle defining a storage volume configured to receive harvested crops, and a sensor assembly provided in association with the crop transport receptacle. The sensor assembly is configured to generate capacitance-related data associated with a fill-level of the harvested crops within the storage volume. In addition, the system includes a computing system communicatively coupled to the sensor assembly. The computing system is configured to receive crop data associated with a dielectric constant of the harvested crops within the storage volume. The computing system is further configured to determine the fill-level of the harvested crops within the storage volume based at least in part on the capacitance-related data and the crop data.

In another aspect, the present subject matter is directed to a system for monitoring crop fill-levels of transport receptacles. The system includes a crop transport receptacle defining a storage volume configured to receive harvested crops, and a sensor assembly provided in association with the crop transport receptacle. The sensor assembly is configured to generate capacitance-related data associated with a fill-level of the harvested crops within the storage volume. The system also includes a secondary fill-level sensor configured to generate data associated with the fill-level of the harvested crops within the storage volume, and a computing system communicatively coupled to the sensor assembly and the secondary fill-level sensor. The computing system is configured to determine a relationship between the capacitance-related data and the fill-level of the harvested crops based at least in part on the data received from the secondary fill-level sensor.

In a further aspect, the present subject matter is directed to a method for monitoring a crop fill-level of a transport receptacle. The method includes receiving, with a computing system, capacitance-related data from a sensor assembly provided within a storage volume of the transport receptacle as harvested crops are being received in the storage volume, the capacitance-related data being associated with a fill-level of the harvested crops within the storage volume. The method also includes receiving, with the computing system, crop data associated with a dielectric constant of the harvested crops within the storage volume, and determining, with the computing system, the fill-level of the harvested crops within the storage volume based at least in part on the capacitance-related data and the crop data.

These and other features, aspects and advantages of the present technology will become better understood with reference to the following description and appended claims. The accompanying drawings, which are incorporated in and constitute a part of this specification, illustrate embodiments of the technology and, together with the description, serve to explain the principles of the technology.

### BRIEF DESCRIPTION OF THE DRAWINGS

A full and enabling disclosure of the present technology, including the best mode thereof, directed to one of ordinary skill in the art, is set forth in the specification, which makes reference to the appended figures, in which:

FIG. 1 illustrates a schematic top view of one embodiment of a harvester and a transport vehicle during an unloading operation in accordance with aspects of the present subject matter;

FIG. 2 illustrates a rear view of one embodiment of a harvester and a transport vehicle during an unloading operation in accordance with aspects of the present subject matter;

FIG. 3 illustrates a schematic view of one embodiment of a system for monitoring the fill-level of a crop transport receptacle in accordance with aspects of the present subject matter;

FIG. 4 illustrates a schematic, cross-sectional view of the crop transport receptacle shown in FIG. 3 taken about line 4-4, particularly illustrating one embodiment of exemplary capacitance-based sensor assemblies that can be used to monitor the fill-level of the crop transport receptacle in accordance with aspects of the present subject matter;

FIG. 5 illustrates a similar schematic, cross-sectional view of the crop transport receptacle as that shown in FIG. 4, particularly illustrating another embodiment of the disclosed system that utilizes secondary fill-level sensors to calibrate the data received from the capacitance-based sensor assemblies in accordance with aspects of the present subject matter; and

FIG. 6 illustrates a flow diagram of one embodiment of a method for monitoring the fill-level of a crop transport receptacle in accordance with aspects of the present subject matter.

Repeat use of reference characters in the present specification and drawings is intended to represent the same or analogous features or elements of the present technology.

### DETAILED DESCRIPTION OF THE DRAWINGS

Reference now will be made in detail to embodiments of the invention, one or more examples of which are illustrated in the drawings. Each example is provided by way of explanation of the invention, not limitation of the invention. In fact, it will be apparent to those skilled in the art that various modifications and variations can be made in the present invention without departing from the scope or spirit of the invention. For instance, features illustrated or described as part of one embodiment can be used with another embodiment to yield still a further embodiment. Thus, it is intended that the present invention covers such modifications and variations as come within the scope of the appended claims and their equivalents.

In general, the present subject matter is directed to systems and methods for monitoring the fill-level of crop transport receptacles, such as the fill-level of a receptacle associated with a transport vehicle that is configured to receive harvested crops from a harvester during the performance of an unloading operation. In several embodiments, the system may include one or more fill-level sensor assemblies provided in association with a crop transport receptacle and a computing system communicatively coupled to each fill-level sensor assembly for monitoring the fill-level of the receptacle based on the data received from the sensor assembly.

In accordance with aspects of the present subject matter, the sensor assemblies of the disclosed system may include one or more capacitance-based fill-level sensor assemblies provided in association with the crop transport receptacle. Each sensor assembly may be configured to generate capacitance-related data associated with a fill-level of harvested crops within the transport receptacle. Additionally, in several embodiments, the computing system may be configured to receive crop data associated with a dielectric constant of the harvested crops (e.g., data related to the crop type and/or moisture content of the harvested crops). In such embodiments, the computing system may be configured to estimate or determine the fill-level of the harvested crops within the transport receptacle based at least in part on the capacitance-related data and the crop data.

Moreover, as an alternative to using the crop data, the computing system may be configured to calibrate the capacitance-related data generated by each sensor assembly using calibration data received from one or more secondary fill-level sensors. For instance, in several embodiments, the system may include at least one secondary fill-level sensor that is configured to generate data associated with a fill-level of the harvested crops within the transport receptacle, with such data being independent of the capacitance-related data generated by each sensor assembly. For instance, the secondary fill-level sensor may correspond to a different type of sensing device, such as a switch-based sensor or a reflection-based sensor. Regardless, the data generated by the secondary fill-level sensor may be used to calibrate the capacitance-related data generated by each sensor assembly, such as by using the data to help establish or determine a relationship between the capacitance-related data and the current fill-level of the crop transport receptacle.

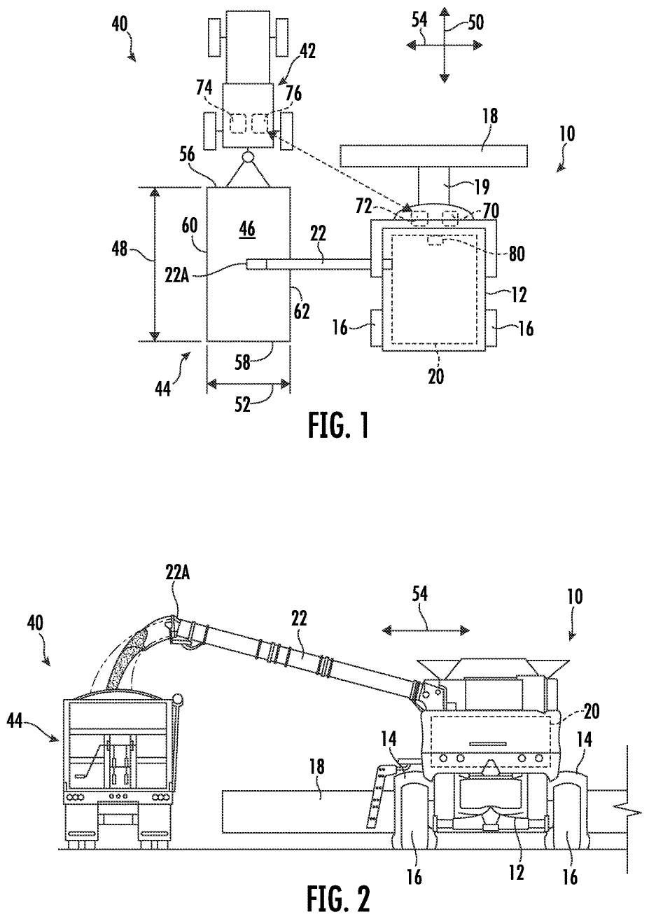

Referring now to FIGS. 1 and 2, respective top and rear views of a harvester 10 and a transport vehicle 40 during the performance of an unloading operation is illustrated in accordance with aspects of the present subject matter. As is generally understood, during a harvesting operation, crops harvested by the harvester 10 can be off-loaded immediately (e.g., in the case of forage harvesters) or temporarily stored within internal storage of the harvester (e.g., in the case of combine harvesters). Regardless, either immediately upon harvesting or after the internal storage is full or substantially full, an unloading operation is performed during which the harvested crops are unloaded from the harvester 10 to a transport vehicle 40. Such an unloading operation can be performed while the vehicles 10, 40 are stationary or can be performed “on-the-go” simultaneously with the performance of a harvesting operation. For instance, for on-the-go unloading operations, the transport vehicle 40 is typically brought into alignment with the harvester 10 such that the harvested crops can be unloaded from the harvester 10 while both vehicles 10, 40 are moving through the field. Such alignment typically includes maintaining desired offset distances between the harvester 10 and the transfer vehicle 40 (e.g., a desired lateral offset distance and/or a desired longitudinal offset distance) to ensure that the harvested crops can be properly unloaded from the harvester 10 and received by the transport vehicle 40.

In the illustrated embodiment, the harvester 10 is configured as a combine, such as an axial-flow type combine or any other suitable type of combine. In such an embodiment, the harvester 10 may include, for example, a chassis 12 and a plurality of ground engaging elements (e.g., front and rear wheels 14, 16) supporting the chassis 12 relative to the ground. In addition, the harvester 10 may include various components coupled to or supported by the chassis 12, including, but not limited to, a header 18, a feeder housing 19, an operator's cab (not shown), various internal crop processing systems and/or sub-systems (e.g., a threshing and separating system, a cleaning system, and/or the like), an internal crop storage tank 20, and an unloading tube or spout 22. The unloading spout 22 may, for example, be configured as an unloading auger, belt conveyor, chain elevator, and/or the like. Regardless of the type, the unloading spout 22 is generally configured to facilitate the transfer of harvested crops from the internal crop storage tank 20 to the transport vehicle 40 during the performance of an unloading operation. In other embodiments, it should be appreciated that the harvester 10 may have any other suitable harvester configuration, such as by being configured as a forage harvester.

In general, the transport vehicle 40 may include both a traction device 42 and a crop transport receptacle 44. As shown in the illustrated embodiment, the traction device 42 corresponds to a work vehicle, namely an agricultural tractor. However, in other embodiments, the traction device 42 may be a truck or other self-propelled vehicle sufficient to carry or tow the transport receptacle 44. Similarly, in the illustrated embodiment, the crop transport receptacle 44 corresponds to a wagon. However, in other embodiments, the transport receptacle 44 may be a grain cart, bin, or other similar storage/transport receptacle. In another embodiment, the transport vehicle 40 may be a semi-trailer truck, tractor-trailer or other similar self-propelled container vehicle.

As particularly shown in FIG. 1, the crop transport receptacle 44 may generally be configured to define a storage volume 46 for receiving/storing harvested crops. For instance, in the illustrated embodiment, the storage volume 46 has a length 48 extending in a longitudinal direction 50 between opposed front and rear walls 56, 58 of the transport receptacle 44 and a width 52 extending in a lateral direction 54 between opposed first and second sidewalls 60, 62 of the transport receptacle 44. During the performance of an unloading the operation, the transport vehicle 40 is generally configured to be aligned relative to the harvester 10 such that harvested crops contained within the internal storage tank 20 of the harvester 10 can be directed through the unloading spout 22 and expelled therefrom into the storage volume 46 of the crop transport receptacle 44. Specifically, a discharge end 22A of the unloading spout 22 may generally be aligned with the transport receptacle 44 in the longitudinal and lateral directions 50, 54 such that harvested crops expelled from the spout 22 are received within the storage volume 46 of the receptacle 44. In this regard, to maintain the desired relative positioning between the discharge end 22A of the unloading spout 22 and the transport receptacle 44 during an on-the-go unloading operations, various aspects of the operation of one or both of the vehicles 10, 40 can be manually or automatically controlled/adjusted, such as by adjusting the speed and/or steering of the harvester 10 and/or the transport vehicle 40. In addition, the unloading spout 22 can be actuated to adjust the position/orientation of the spout 22 relative to the transport receptacle 44, such as by actuating the spout 22 to adjust the longitudinal position of the discharge end 22A relative to the front and rear walls 56, 58 of the receptacle 44 and/or to adjust the lateral position of the discharge end 22A relative to the first and second sidewalls 60, 62 of the receptacle 44.

In several embodiments, the harvester 10 and/or the transport vehicle 40 may include a moisture sensor configured to generate data associated with the moisture content of the harvested crops. For instance, in the illustrated embodiment, the harvester 10 is shown as including a moisture sensor 80 (FIG. 1) for generating data associated with the moisture content of the harvested crops being processed by and/or stored within the harvester 10. However, in other embodiments, the transport vehicle 40 may alternatively (or additionally) include a moisture sensor 80 configured to generate data indicative of the moisture content of the crops contained within the transport receptacle 44. It should be appreciated that the moisture sensor 80 may generally correspond to any suitable moisture sensing device, such as a capacitance-based or resistance-based moisture sensor.

Additionally, in several embodiments, both the harvester 10 and the transport vehicle 40 may include on-board computing systems and associated wireless communications devices. For instance, as shown in FIG. 1, the harvester 10 may include a harvester-based computing system 70 and wireless communications device 72, while the transport vehicle 40 may include a transport-based computing system 74 and wireless communications device 76. In such embodiments, the vehicles 10, 40 may be equipped for vehicle-to-vehicle communications, for example, by allowing data, including information, requests, instructions, control signals, and/or the like, to be transmitted between the on-board computing systems 70, 74 via the associated wireless communications devices 72, 76. Such data may, for instance, correspond to sensor data associated with the fill-level of the crop transport receptacle 44, including data associated with the overall fill-level of the transport receptacle 44 and/or data associated with the fill-level of individual zones or regions of the transport receptacle 44. In addition, the data may include, for example, sensor data associated with the moisture content of the harvested crops (e.g., from moisture sensor 80) and/or data associated with the type of crop being harvested.

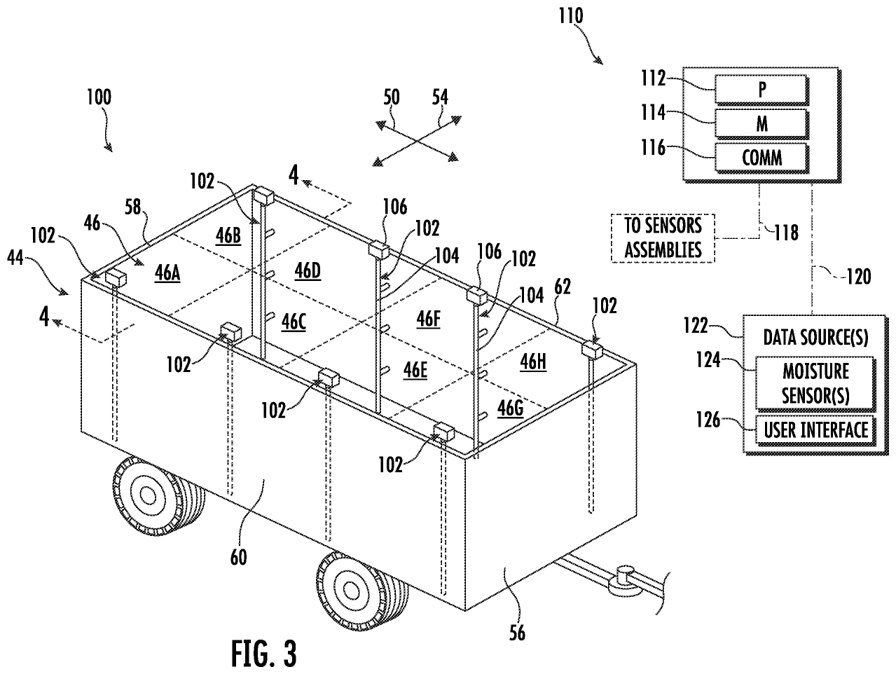

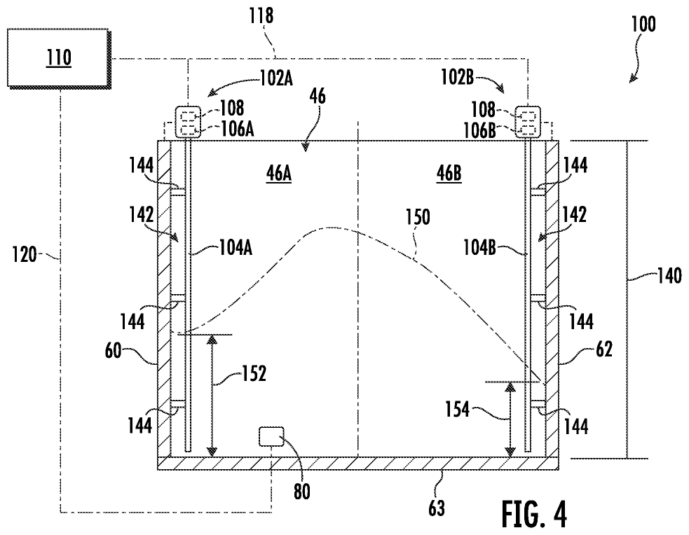

Referring now to FIG. 3, a schematic view of one embodiment of a system 100 for monitoring the fill-level of a crop transport receptacle is illustrated in accordance with aspects of the present subject matter. For purposes of discussion, the system 100 will generally be described with reference to the crop transport receptacle 44 and related transport vehicle 40 shown in FIGS. 1 and 2. However, in other embodiments, the disclosed system 100 may be configured for use with transport receptacles having any other suitable configuration, including transport receptacles provided in association with any other suitable traction device and/or forming part of any other suitable transport vehicle.

As shown in FIG. 3, the system 100 includes a crop transport receptacle (e.g., the receptacle 44 described above with reference to FIGS. 1 and 2) and one or more fill-level sensor assemblies 102 provided in operative association with the transport receptacle 44. In general, each sensor assembly 102 is configured to generate data associated with the fill-level of all or a portion of the receptacle 44. For instance, in one embodiment, each sensor assembly 102 may be configured to generate data associated with the overall fill-level of the receptacle 44. In addition to such data (or as an alternative thereto), each sensor assembly 102 may be configured to generate data associated with a localized fill-level of the receptacle 44, such as the fill-level within a given region or zone of the receptacle 44. For example, the storage volume 46 of the transport receptacle 44 may, in certain embodiments, be sub-divided into separate zones or regions. In such embodiments, the fill-level of each individual zone or region defined within the transport receptacle 44 may be monitored via one or more respective sensor assemblies 102.

In several embodiments, each sensor assembly 102 may correspond to a capacitance-based sensor assembly configured to generate data indicative of the fill-level of the transport receptacle 44 based on a monitored capacitance value. For instance, as will be described below, each sensor assembly 102 may include a sensor probe 104 supported relative to an adjacent wall of the transport receptacle 44 (e.g., one of receptacle walls 56, 58, 60, 62) and a sensor 106 configured to measure a capacitance or a parameter indicative of the capacitance across the sensor probe 104 and the adjacent wall. Specifically, the sensor probe 104 may be positioned at a given distance from the adjacent wall along the vertical height of the wall such that harvested crops can accumulate vertically in a gap defined between the probe 104 and the adjacent wall as the crops begin to fill-up the receptacle 44. Additionally, the sensor probe 104 may be supported relative to the adjacent wall in the manner that electrically isolates the probe 104 from the wall (e.g., by using electrically isolating mounts). As a result, when a voltage is applied to the probe/wall, an electric field is generated between the probe/wall (e.g., similar to a parallel plate capacitator). In general, the measured capacitance between the probe/wall will increase as more harvested crops accumulate within the gap defined therebetween. As such, by knowing a correlation between the measured capacitance and the vertical height along which the harvested crops extend within the gap defined between the probe/wall (e.g., via look-up tables), the current fill-level of the storage volume 46 may be estimated or determined.

As particularly shown in the illustrated embodiment, the system 100 includes a plurality of sensor assemblies 102 spaced apart from one another within the storage volume 46 of the transport receptacle 44. Specifically, as shown in FIG. 3, the system 100 includes four sensor assemblies 102 positioned within the storage volume 46 at a location adjacent to each sidewall 60, 62 of the receptacle 44, with the sensor assemblies 102 being spaced apart from one another along the adjacent wall 60, 62 in the longitudinal direction 50 of the receptacle 44. In such an embodiment, each sensor assembly 102 may be configured to generate data indicative of the fill-level of a respective storage region or zone of the storage volume 46. For instance, in the illustrated embodiment, the storage volume 46 has been sub-divided into eight separate storage zones (e.g., zones 46A-46H) corresponding to the locations of the eight sensor assemblies 102, thereby permitting the fill-level of each individual storage zone to be monitored based on the data received from the respective sensor assembly 102. It should be appreciated that, in other embodiments, the system 100 may include any other suitable number of sensor assemblies 102, such as seven or less sensor assemblies or nine or more sensor assemblies, as well as any suitable number of storage zones within which the localized fill-level of the storage volume 46 may be monitored.

Additionally, as shown in FIG. 3, the system 100 may also include a computing system 110 communicatively coupled to each sensor assembly 102. In general, the computing system 110 may be configured to monitor the fill-level of the crop transport receptacle 44 based on data received from the sensor assemblies 102 (e.g., data indicative of the measured capacitance between the sensor probe 104 and the adjacent wall). For instance, as will be described below, the computing system 110 may include suitable algorithms, mathematical formulas or expressions, predetermination relationships, correlation tables, look-up tables, and/or other data stored within its memory that allows the computing system 110 to determine, calculate, or estimate the fill-level within all or a portion of the receptacle 44 based on the data received from the sensor assemblies 102.

In general, the computing system 110 may comprise any suitable processor-based device known in the art, such as a computing device or any suitable combination of computing devices. Thus, in several embodiments, the computing system 110 may include one or more processor(s) 112 and associated memory device(s) 114 configured to perform a variety of computer-implemented functions. As used herein, the term “processor” refers not only to integrated circuits referred to in the art as being included in a computer, but also refers to a controller, a microcontroller, a microcomputer, a programmable logic controller (PLC), an application specific integrated circuit, and other programmable circuits. Additionally, the memory device(s) 114 of the computing system 110 may generally comprise memory element(s) including, but not limited to, a computer readable medium (e.g., random access memory (RAM)), a computer readable non-volatile medium (e.g., a flash memory), a floppy disk, a compact disc-read only memory (CD-ROM), a magneto-optical disk (MOD), a digital versatile disc (DVD) and/or other suitable memory elements. Such memory device(s) 114 may generally be configured to store suitable computer-readable instructions that, when implemented by the processor(s) 112, configure the computing system 110 to perform various computer-implemented functions, such as one or more aspects of the methods or algorithms described herein.

In addition, the computing system 110 may also include various other suitable components, such as a communications circuit or module, one or more input/output channels, a data/control bus and/or the like. For instance, the computing system 110 may include a communications module or interface 116 to allow the computing system 110 to communicate with any of the various other system components described herein, such as the sensor assemblies 102. Specifically, as shown schematically in FIG. 3, the communications module 116 may be communicatively coupled to each sensor assembly 102 via one or more communicative links 118 to allow data to be transmitted from the sensor assemblies 102 to the computing system 110. Additionally, as shown in FIG. 3, the computing system 110 may also be communicative coupled to one or more additional data sources 122 via one or more communicative links 120 to allow data to be transmitted from such data sources 122 to the computing system 110. For instance, in one embodiment, the computing system 110 may be communicatively coupled to a moisture sensor (e.g., moisture sensor 80) configured to provide data indicative of the moisture content of the harvested crops being received within the transport receptacle 44. As described above, the moisture sensor 80 may be provided in association with the harvester 10 to allow the moisture content of the harvested crops being processed through or stored within the harvester 10 to be measured and/or the moisture sensor 80 may be provided in association with the transport vehicle 40 to allow the moisture content of the harvested crops received within the transport receptacle to be measured. In embodiments in which the moisture sensor 80 is provided in association with the harvester 10, the computing system 110 may, for instance, be communicatively coupled to the moisture sensor 80 via the on-board computing system 70 of the harvester 10 (e.g., by receiving moisture sensor data from the computing system 70 via the associated wireless communications device 72). Moreover, as shown in FIG. 3, the computing system 110 may also be communicatively coupled to a user interface 126 to allow data input from the operator of the harvester 10 and/or the transport vehicle 40 to be communicated to the computing system 110. For instance, in several embodiments, the computing system 110 may receive data associated with the type of the crops being received within the transport receptacle 44 (e.g., wheat, oats, barley, rice, and other grains, such as cereals, legumes, etc.) via the user interface 126 (e.g., by receiving such data directly or indirectly via a separate computing system, such as the on-board computing system 70 of the harvester 10).

It should be appreciated that, in several embodiments, the computing system 110 may correspond to a stand-alone computing system configured to monitor the fill-level of the crop transport receptacle 44. In such embodiments, the computing system 110 may, for instance, be configured to communicate data related to the fill-level of the transport receptacle 44 to one or more separate computing systems, such as by communicating the data to the on-board computing system of an associated transport vehicle and/or harvester (e.g., on-board computing systems 70, 74 shown in FIG. 1). Additionally, in some embodiments, the computing system 110 may correspond to or form part of an existing on-board computing system, such as the on-board computing system of an associated transport vehicle (e.g., computing system 74 (FIG. 1)).

As indicated above, the computing system 110 may include suitable algorithms, mathematical formulas or expressions, predetermination relationships, correlation tables, look-up tables, and/or other data stored within its memory that allows the computing system 110 to determine, calculate, or estimate the fill-level within all or a portion of the receptacle 44 based on the data received from the sensor assemblies 102. Specifically, in several embodiments, the computing system 110 may include a plurality of look-up tables that correlate the crop type (e.g., as determined based on data input via the user interface 126), the moisture content of the harvested crops (e.g., as determined based on data received from the moisture sensor 80), and the measured capacitance value (e.g., as determined based on data received from one or more of the sensor assemblies 102) to an associated fill-level of the transport receptacle 44. For instance, in one embodiment, the computing system 110 may store a look-up table for each potential crop type that correlates moisture content to an associated dielectric constant for such crop type. Additionally, the computing system 110 100 may store a look-up table for each potential dielectric constant (or a look-up table for each of a plurality of different ranges or bands of potential dielectric constants) that correlates the measured capacitance value to an associated fill-level (e.g., a fill-height or fill percentage). Thus, by determining the crop type and moisture content of the harvested crops, the computing system 110 may look-up or otherwise identify an associated dielectric constant for the crops currently being received within the transport receptacle. Thereafter, by knowing the dielectric constant value of the crops and by monitoring the capacitance between the sensor probe 104 of a given sensor assembly 102 and the adjacent wall, the computing system 110 may look-up or otherwise identify a fill-level associated with the crops that have accumulated between the probe/wall.

It should be appreciated by those of ordinary skill in the art that the moisture content of the harvested crops may often vary throughout the field during the performance of a harvesting operation. Accordingly, since the dielectric constant of the crops will also vary with variations in the moisture content, the computing system 110 may be configured to account for such variations in the moisture content. For instance, in several embodiments, the computing system 110 may be configured to calculate a running average of the moisture content of the harvested crops and utilize such running average to determine the associated dielectric constant, such as by using the running average within the relevant look-up table to identify the appropriate dielectric constant for the harvested crops.

Moreover, in several embodiments, based on the monitored fill-level of the crop transport receptacle 44, the computing system 110 may be configured to initiate one or more control actions during the performance of an unloading operation to adjust the operation of a related transport vehicle and/or harvester (e.g., the transport vehicle 40 and/or harvester 10 described above with reference to FIGS. 1 and 2). For instance, it may be desirable to fill the crop transport receptacle 44 according to a predetermined filling strategy, such as by filling the receptacle 44 front-to-back in the longitudinal direction 50, side-to-side on the lateral direction 54, or according to a zone-based filling scheme. In such instance, to implement the desired filling strategy, the relative position between the transport receptacle 44 and the unloading spout 22 (FIGS. 1 and 2) of the harvester 10 may be adjusted as different sections or zones of the receptacle 44 begin to fill-up (e.g., as monitored via the data from the fill-level sensor assemblies 102). In one embodiment, a transport-based control action may be executed to adjust the relative position between the transport receptacle 44 and the unloading spout 22, such as by adjusting the speed of the transport vehicle 40 (e.g., speeding up or slowing down) or by adjusting the heading of the transport vehicle 40 (e.g., by steering the vehicle 40 left or right relative to the harvester 10). In another embodiment, a harvester-based control action may be executed to adjust the relative position between the transport receptacle 44 and the unloading spout 22, such as by adjusting the speed of the harvester 10 (e.g., speeding up or slowing down), by adjusting the heading of the harvester 10 (e.g., by steering the harvester 10 left or right relative to the vehicle 10), or by implementing spout control to acuate the spout 22 relative to the remainder of the harvester 10.

It should be appreciated that the computing system 110 may be configured to initiate control actions to adjust the relative position between the transport receptacle 44 and the unloading spout 22 in any suitable manner. For instance, in one embodiment, control actions may be initiated by transmitting the fill-level sensor data (or the current fill-level as determined based on the sensor data) from the computing system 110 to a separate computing system (e.g., the on-board computing system(s) 70, 74 of the transport vehicle 40 and/or the harvester 10), at which point the separate computing system may be configured to process/analyze the sensor data and transmit control signals for executing a suitable control action(s) to make a desired adjustment(s) in the relative positioning between the transport receptacle 44 and the unloading spout 22, including the transmission of control signals associated with instructions or requests for executing the desired adjustments. In other embodiments, the computing system 110 may be configured to process/analyze the sensor data and subsequently transmit, itself, control signals for executing a suitable control action(s) to make a desired adjustment(s) in the relative positioning between the transport receptacle 44 and the unloading spout 22, including the transmission of control signals associated with instructions or requests for executing the desired adjustments.

Referring now to FIG. 4, a schematic, cross-sectional view of a portion of the crop transport receptacle 44 shown in FIG. 3 taken about line 4-4 is illustrated in accordance with aspects of the present subject matter, particularly illustrating exemplary fill-level sensor assemblies 102 (e.g., first and second sensor assemblies 102A, 102B) for monitoring the fill-level of the receptacle 44. As indicated above, each sensor assembly 102 may generally include a sensor probe 104 supported relative to an adjacent wall of the transport receptacle 44. For instance, as shown in the illustrated embodiment, the first sensor assembly 102A includes a first sensor probe 104A supported adjacent to the first sidewall 60 of the transport receptacle 44 such that the sensor probe 104A extends lengthwise generally parallel to the adjacent sidewall 60 along all or a substantially all of a vertical height 140 of the storage volume 46 of the receptacle 44 (e.g., from a location adjacent the open end of the receptacle 44 to a location adjacent a bottom wall 63 of the receptacle 44). Similarly, the second sensor assembly 102B includes a second sensor probe 104B supported adjacent to the second sidewall 62 of the transport receptacle 44 such that the sensor probe 104B extends lengthwise generally parallel to the adjacent sidewall 62 along all or a substantially all of the vertical height 140 of the storage volume 46 of the receptacle 44 (e.g., from a location adjacent the open end of the receptacle 44 to a location adjacent the bottom wall 63 of the receptacle 44). As shown in FIG. 4, each sensor probe 104A, 104B may be supported relative to its respective adjacent wall 60, 62 in a spaced apart relationship such that a lateral gap 142 is defined between each probe/wall. For instance, in the illustrated embodiment, each sensor probe 104A, 104B is supposed relative to its respective adjacent wall 60, 62 via spacer mounts 144 coupled between the probe 104A, 104B and the adjacent wall 60, 62. The spacer mounts 144 may, in several embodiments, be formed from a non-conducting material such that the mounts 144 electrically isolate each probe 104A, 104B from its respective adjacent wall 60, 62.

Additionally, as indicated above, each sensor assembly 102 may include a sensor 106 configured to measure a capacitance or a parameter indicative of the capacitance across the adjacent probe/wall. For instance, as shown in FIG. 4, the first sensor assembly 102A includes a first capacitance sensor 106A and the second sensor assembly 102B includes a second capacitance sensor 106B. In general, the sensors 106A, 106B may be configured as any suitable sensing device for measuring a parameter indicative of capacitance. For instance, in one embodiment, each sensor 106A, 106B may correspond to a capacitance meter such that the sensor directly measures the capacitance across the respective probe/wall. Alternatively, each sensor 106A, 106B may configured to measure any other parameter indicative of capacitance, such as voltage, and/or the like. Additionally, each sensor 106A, 106B may be configured to transmit data indicative of the measured capacitance between the respective adjacent probe/wall to the computing system 110 for subsequent processing and analysis (e.g., via link 118).

By configuring the sensor assemblies 102A, 102B as described herein, each sensor probe 104A, 104B and its respective adjacent receptacle wall 60, 62 may function similar to a parallel-plate capacitor. Thus, when a voltage is applied to the probe/wall (e.g., via a suitable voltage source or voltage supply 108 associated with the sensor assembly 102), an electric field is generated between the probe/wall. As indicated above, the measured capacitance between each probe/wall will generally increase as more harvested crops accumulate vertically within the lateral gap 142 defined therebetween. As such, by knowing a correlation between the measured capacitance and the vertical height along which the harvested crops extend within the gap 142 defined between the probe/wall (e.g., via look-up tables), the current fill-level of the storage volume 46 may be estimated or determined. For instance, a representative crop material fill line 150 is illustrated in FIG. 4 providing an example fill-level within the crop transport receptacle 44. With such a fill-level, crops have accumulated within the lateral gap 142 defined between the first sensor probe 104A and the adjacent receptacle wall 60 up to a first vertical height 152 while crops have accumulated within the lateral gap 142 defined between the second sensor probe 104B and the adjacent receptacle wall 62 up to a second vertical height 154. By measuring the capacitance between the first sensor probe 104A and the adjacent receptacle wall 60, the measured capacitance can be corelated to an associated fill-level value (e.g., height 152) for the respective sub-region 46A of the receptacle 44. Similarly, by measuring the capacitance between the second sensor probe 10BA and the adjacent receptacle wall 62, the measured capacitance can be corelated to an associated fill-level value (e.g., height 154) for the respective sub-region 46B of the receptacle 44.

In general, the relationship between the measured capacitance and the fill-level of the transport receptacle 44 will generally vary as a function of the dielectric constant of the harvested crops contained within the receptable 44. Thus, to correlate the measured capacitance to an associated fill-level, the computing system 110 may, in several embodiments, be configured to receive crop data associated with the dielectric constant of the harvested crops, such as information related to the crop type (e.g., wheat, oats, barley, rice, other grains, such as cereals, legumes, etc.) and/or information related to the moisture content of the crops. As indicated above, information related to the crop type may, in one embodiment, be received from the operator via an associated user interface. In another embodiment, the computing system 110 may be communicatively coupled to a suitable sensor or other data source to obtain data associated with the crop type. Additionally, as indicated above, information related to the moisture content may be received from a moisture sensor 80, such as a harvester-based moisture sensor or a transport-based moisture sensor. For instance, in the embodiment shown in FIG. 4, the computing system 110 is communicatively coupled (e.g., via link 120) to a moisture sensor 80 provided within the crop transport receptacle 44 to generate data associated with the moisture content of the harvested crops. Alternatively, as described above with reference to FIG. 1, the moisture sensor 80 may be provided within the harvester 10, in which case the moisture data may be transmitted to the computing system 110 from a separate computing system, such as the on-board computing system 70 of the harvester 10. Regardless of the source of the information, the data associated with the crop type and moisture content may be used to determine a dielectric constant of the harvested crops (e.g., via a look-up table), which may then be used in combination with the measured capacitance to determine an associated fill-level of the crop transport receptacle (e.g., via another look-up table).

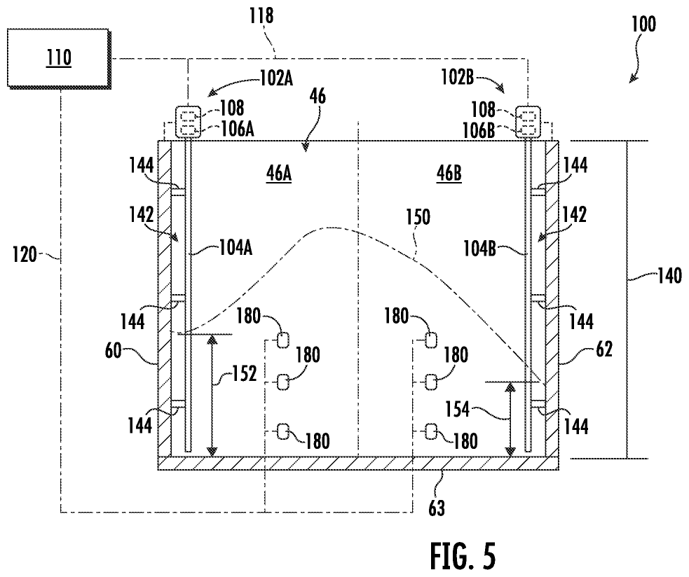

As an alternative to determining or estimating the dielectric constant of the harvested crops (e.g., using the associated crop data), the capacitance-related data from the sensor assemblies 102 can be correlated to an associated fill-level of the crop transport receptacle using calibration data from one or more secondary fill-level sensors. For instance, FIG. 5 illustrates a similar schematic, cross-sectional view of the portion of the crop transport receptacle 44 shown in FIG. 4, particularly illustrating an alternative embodiment of the disclosed system 100 in which secondary fill-level sensors are used to calibrate the data generated by the sensor assemblies 102.

As shown in FIG. 5, in addition to the sensor assemblies 102, the system 100 includes one or more secondary fill-level sensors 180 provided in association with the crop transport receptacle 44. In several embodiments, each secondary fill-level sensor 180 may correspond to a switch-based fill-level sensor positioned within the interior of the crop transport receptacle 44. For instance, each secondary fill-level sensor 180 may be configured as a contact-based pressure switch positioned on an inner surface of the crop transport receptacle 44, such as the inner surface defined by one or more of the walls 56, 68, 60, 62 of the crop transport receptacle 44. In such an embodiment, by positioning each switch-based fill-level sensor 180 at a given location within the crop transport receptacle 44, the sensor 180 may be configured to detect when harvested crops begin to accumulate within the receptacle 44 at or adjacent to the location of the sensor 180. For instance, when the harvested crops contact or push/press against a secondary fill-level sensor 180 as the crops accumulate at or adjacent to the sensor 180, an internal circuit of the switch-based sensor will close (or open), thereby providing a signal (or a lack thereof) to the computing system 110 (e.g., via link 120) as an indicator that the harvested crops have reached the level or vertical height of the sensor 180 within the receptacle 44. As shown in FIG. 5, in one embodiment, an array of switch-based fill-level sensors 180 may be provided in association with each sub-region 46A, 46B (e.g., a vertical column of sensors 180), thereby allowing the fill-level within the sub-region 46A, 46B to be determined at multiple different heights or fill-levels.

It should be appreciated that, as an alternative to switch-based sensors, the secondary fill-level sensor(s) 180 may correspond to any other suitable sensor or sensing device configured to provide an indication of the fill-level within the crop transport receptacle 44. For instance, in an alternative embodiment, the secondary fill-level sensor(s) 180 may correspond to one or more non-contact, reflection-based fill-level sensors, such as one or more radar sensors, sonar sensors, ultrasound sensors, LIDAR sensors, and/or the like. In such an embodiment, each secondary fill-level sensor 180 may be supported relative to the transport receptacle 44 such that the sensor 180 is configured to transmit waves towards the harvested crops accumulating within the transport receptacle 44 and subsequently detect the return waves as reflected off the top surface of the accumulated crops to determine the distance between the sensor 180 and the top surface of the accumulated crops, which can then be used to calculate or estimate the fill-level of the receptacle 44.

By providing the secondary fill-level sensors 180, the capacitance-related data provided by the sensor assemblies 102 may be calibrated without requiring any information related to the dielectric constant of the harvested crops. For instance, in the illustrated embodiment, each switch-based secondary fill-level sensor 180 may be configured to indicate when the fill-level within a given sub-region 46A, 46B of the transport receptacle 44 reaches a known or threshold fill-level within such sub-region 46A, 46B corresponding the height of the sensor 180 with the receptable 44. Upon receiving data from a given secondary fill-level sensor 180 indicating that the fill-level has reached the known or threshold level associated with the height of such sensor 180, the computing system 110 may correlate the measured capacitance for the corresponding sensor assembly 102 with such fill-level or vertical height. Each time a subsequent secondary fill-level sensor 180 is triggered (e.g., each higher sensor 180 within the vertical array), the computing system 110 may be configured to confirm and/or update the correlation between the measured capacitance for the corresponding sensor assembly 102 and the current fill-level within the associated sub-region 46A, 46B. This correlation may be used, for instance, to select an appropriate look-up table relating the measure capacitance to the current fill-level within the receptacle 44 or to establish a mathematical function or expression relating the measure capacitance to the current fill-level based on the calibration values determined using the secondary fill-level sensors 180.

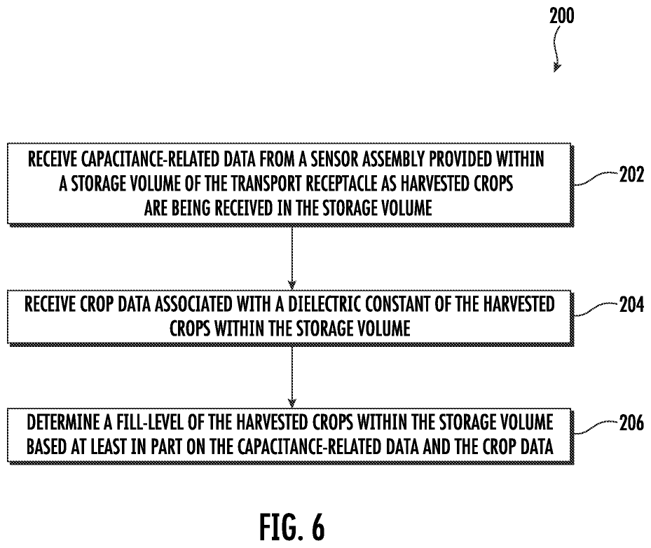

Referring now to FIG. 6, a flow diagram of one embodiment of a method 200 for monitoring the fill-level of a crop transport receptacle is illustrated in accordance with aspects of the present subject matter. In general, the method 200 will be described herein with reference to the crop transport receptacle 44 and system 100 described above with reference to FIGS. 1-5. However, it should be appreciated by those of ordinary skill in the art that the disclosed method 200 may generally be utilized in association with transport receptacles having any suitable receptacle configuration and/or with systems having any other suitable system configuration. In addition, although FIG. 6 depicts steps performed in a particular order for purposes of illustration and discussion, the methods discussed herein are not limited to any particular order or arrangement. One skilled in the art, using the disclosures provided herein, will appreciate that various steps of the methods disclosed herein can be omitted, rearranged, combined, and/or adapted in various ways without deviating from the scope of the present disclosure.

As shown in FIG. 6, at (202), the method 200 may include receiving capacitance-related data from a sensor assembly provided within a storage volume of the transport receptacle as harvested crops are being received in the storage volume. For instance, as indicated above, the computing system 110 may be communicatively coupled to one or more capacitive-based sensor assemblies 102 configured to generate capacitance-related data associated with a fill-level of the harvested crops within the transport receptacle 44.

Additionally, at (204), the method 200 may include receiving crop data associated with a dielectric constant of the harvested crops within the storage volume. For instance, as indicated above, the computing system 110 may be configured to receive data related to the crop type and/or the moisture content of the harvested crops, both which are associated with the dielectric constant of the harvested crops. associated with the

Moreover, at (206), the method 200 may include determining a fill-level of the harvested crops within the storage volume based at least in part on the capacitance-related data and the crop data. For instance, as indicated above, in one embodiment, the computing system 110 may be configured to estimate or determine the fill-level using one or more look-up tables that correlate the crop data and the capacitance-related data to the current fill-level of the transport receptacle 44.

It should also be appreciated that the disclosed method 200 may also include initiating a control action to adjust a relative position between an unloading spout of an associated harvester and the transport receptacle based on the monitored fill-level of the plurality of different storage zones. For instance, as indicated above, the computing system 110 may be configured to initiate a control action to adjust the relative positioning between the spout 22 and the receptacle 44 to ensure a desired distribution of the harvested crops within the receptacle 44 and/or to follow a predetermined fill strategy for the receptacle 44.

It is to be understood that the steps of the method 200 are performed by the computing system 110 upon loading and executing software code or instructions which are tangibly stored on a tangible computer readable medium, such as on a magnetic medium, e.g., a computer hard drive, an optical medium, e.g., an optical disc, solid-state memory, e.g., flash memory, or other storage media known in the art. Thus, any of the functionality performed by the computing system 110 described herein, such as the method 200, is implemented in software code or instructions which are tangibly stored on a tangible computer readable medium. The computing system 110 loads the software code or instructions via a direct interface with the computer readable medium or via a wired and/or wireless network. Upon loading and executing such software code or instructions by the computing system 110, the computing system 110 may perform any of the functionality of the computing system 110 described herein, including any steps of the method 200 described herein.

The term “software code” or “code” used herein refers to any instructions or set of instructions that influence the operation of a computer or controller. They may exist in a computer-executable form, such as machine code, which is the set of instructions and data directly executed by a computer's central processing unit or by a controller, a human-understandable form, such as source code, which may be compiled in order to be executed by a computer's central processing unit or by a controller, or an intermediate form, such as object code, which is produced by a compiler. As used herein, the term “software code” or “code” also includes any human-understandable computer instructions or set of instructions, e.g., a script, that may be executed on the fly with the aid of an interpreter executed by a computer's central processing unit or by a controller.

This written description uses examples to disclose the technology, including the best mode, and also to enable any person skilled in the art to practice the technology, including making and using any devices or systems and performing any incorporated methods. The patentable scope of the technology is defined by the claims, and may include other examples that occur to those skilled in the art. Such other examples are intended to be within the scope of the claims if they include structural elements that do not differ from the literal language of the claims, or if they include equivalent structural elements with insubstantial differences from the literal language of the claims.

1. A system for monitoring crop fill-levels of transport receptacles, the system comprising:
a crop transport receptacle defining a storage volume configured to receive harvested crops;
a sensor assembly provided in association with the crop transport receptacle and being configured to generate capacitance-related data associated with a fill-level of the harvested crops within the storage volume; and
a computing system communicatively coupled to the sensor assembly, the computing system being configured to receive crop data associated with a dielectric constant of the harvested crops within the storage volume, the computing system being further configured to determine the fill-level of the harvested crops within the storage volume based at least in part on the capacitance-related data and the crop data.

2. The system of claim 1, wherein the sensor assembly comprises a sensor probe extending parallel to an adjacent wall of the crop transport receptacle and being spaced apart from the adjacent wall such that a gap is defined between the sensor probe and the adjacent wall along a vertical height of the storage volume within which the harvested crops can accumulate vertically as the fill-level of the harvested crops within the storage volume increases.

3. The system of claim 2, wherein the capacitance-related data comprises data that is indicative of a capacitance between the sensor probe and the adjacent wall when a voltage is applied thereto, the capacitance varying as a function of an amount of the harvested crops positioned within the gap defined between the sensor probe and the adjacent wall along the vertical height of the storage volume.

4. The system of claim 3, wherein the computing system is configured to determine the fill-level of the harvested crops within the storage volume using a look-up table that correlates the fill-level and the capacitance between the sensor probe and the adjacent wall as a function of the dielectric constant of the harvested crops.

5. The system of claim 2, wherein the sensor probe is supported relative to the adjacent wall via spacer mounts, the spacer mounts being configured to electrically isolate the sensor probe from the adjacent wall.

6. The system of claim 1, wherein the crop data comprises at least one of data related to a crop type of the harvested crops or data related to a moisture content of the harvested crops.

7. The system of claim 6, wherein the computing system is configured to receive the data related to the moisture content from a moisture sensor that is communicatively coupled to the computing system or from separate computing system that is communicatively coupled to the moisture sensor.

8. The system of claim 6, wherein the computing system is configured to receive the crop data, at least in part, from a separate computing system.

9. A system for monitoring crop fill-levels of transport receptacles, the system comprising:
a crop transport receptacle defining a storage volume configured to receive harvested crops;
a sensor assembly provided in association with the crop transport receptacle and being configured to generate capacitance-related data associated with a fill-level of the harvested crops within the storage volume;
a secondary fill-level sensor configured to generate data associated with the fill-level of the harvested crops within the storage volume; and
a computing system communicatively coupled to the sensor assembly and the secondary fill-level sensor, the computing system being configured to determine a relationship between the capacitance-related data and the fill-level of the harvested crops based at least in part on the data received from the secondary fill-level sensor.

10. The system of claim 9, wherein the secondary fill-level sensor is configured to generate data indicative of a current fill height of the harvested crops within the storage volume, the computing system being configured to correlate the current fill height to the capacitance-related data to determine the relationship between the capacitance-related data and the fill-level of the harvested crops.

11. The system of claim 9, wherein the secondary fill-level sensor comprises a switch-based sensor or a reflection-based sensor.

12. The system of claim 9, wherein the sensor assembly comprises a sensor probe extending parallel to an adjacent wall of the crop transport receptacle and being spaced apart from the adjacent wall such that a gap is defined between the sensor probe and the adjacent wall along a vertical height of the storage volume within which the harvested crops can accumulate vertically as the fill-level of the harvested crops within the storage volume increases.

13. The system of claim 12, wherein the capacitance-related data comprises data indicative of a capacitance between the sensor probe and the adjacent wall when a voltage is applied thereto, the capacitance varying as a function of an amount of the harvested crops positioned within the gap defined between the sensor probe and the adjacent wall along the vertical height of the storage volume.

14. The system of claim 12, wherein the sensor probe is supported relative to the adjacent wall via spacer mounts, the spacer mounts being configured to electrically isolate the sensor probe from the adjacent wall.

15. A method for monitoring a crop fill-level of a transport receptacle, the method comprising:
receiving, with a computing system, capacitance-related data from a sensor assembly provided within a storage volume of the transport receptacle as harvested crops are being received in the storage volume, the capacitance-related data being associated with a fill-level of the harvested crops within the storage volume;
receiving, with the computing system, crop data associated with a dielectric constant of the harvested crops within the storage volume; and
determining, with the computing system, the fill-level of the harvested crops within the storage volume based at least in part on the capacitance-related data and the crop data.

16. The method of claim 15, wherein the harvested crops are being received in the storage volume of the transport receptacle from a harvester during the performance of an unloading operation, the method further comprising initiating a control action to adjust a relative position between an unloading spout of the harvester and the transport receptacle based on the determined fill-level of the harvested crops within the storage volume.

17. The method of claim 16, wherein the sensor assembly comprises a sensor probe extending parallel to an adjacent wall of the crop transport receptacle and being spaced apart from the adjacent wall such that a gap is defined between the sensor probe and the adjacent wall along a vertical height of the storage volume within which the harvested crops can accumulate vertically as the fill-level of the harvested crops within the storage volume increases;
wherein receiving the capacitance-related data comprises receiving data indicative of a capacitance between the sensor probe and the adjacent wall when a voltage is applied thereto, the capacitance varying as a function of an amount of the harvested crops positioned within the gap defined between the sensor probe and the adjacent wall along the vertical height of the storage volume.

18. The method of claim 17, wherein determining the fill-level of the harvested crops within the storage volume comprises determining the fill-level of the harvested crops within the storage volume using a look-up table that correlates the fill-level and the capacitance between the sensor probe and the adjacent wall as a function of the dielectric constant of the harvested crops.

19. The method of claim 15, wherein receiving the crop data comprises receiving at least one of data related to a crop type of the harvested crops or data related to a moisture content of the harvested crops.

20. The method of claim 15, wherein receiving the crop data comprises receiving the crop data, at least in a part, from a separate computing system.
